(19) United States
(12) Patent Application Publication
(10) Pub. No.: US 2002/0048557 A1
(43) Pub. Date: Apr. 25, 2002

(54) Antiperspirants and deodorants with low white residue on skin and fabric

(76) Inventors: Heng Cai, Yardley, PA (US); Aixing Fan, Bridgewater, NJ (US); Anthony Esposito, Roselle, NJ (US)

Correspondence Address: COLGATE-PALMOLIVE COMPANY, 909 River Road, P.O. Box 1343, Piscataway, NJ, 08855-1343, US

(21) Appl. No.: 09/922,091

(22) Filed: Aug. 3, 2001

Related U.S. Application Data
(60) Provisional application No. 60/229,444, filed on Aug. 31, 2000.

(51) Int. Cl. A61K 7/32

(52) U.S. Cl. 424/65

(57) ABSTRACT

Antiperspirant and/or deodorant compositions are disclosed that exhibit low white residue on both skin and fabric and which are formed by combining selected silicone polyamides with a selected co-gellant and an antiperspirant active having a high bulk density. The compositions may be formed as sticks, creams or roll-ons with good aesthetics.

This invention claims priority under 35 USC 119(e)(1) based on Provisional application Ser. No. 60/229,444, filed Aug. 31, 2000.

### FIELD OF THE INVENTION

This invention relates to antiperspirant and/or deodorant products that are made with siloxane-based polyamides and co-gellants, and exhibit low white residue on both skin and black fabrics. Historically it has been a focus of the art to achieve products having low residue on skin, but having substantially low white residue on black fabrics (as test standard) remained as an unmet performance standard. This invention solves this problem by using siloxane-based polyamide and co-gellant systems in combination with a high-density active ingredient. The products may be characterized as including opaque suspensions and translucent to opaque emulsions, and are formulated as sticks, soft solids, gels or roll-ons.

### BACKGROUND OF THE INVENTION

The present invention is directed to improved antiperspirant and/or deodorant products formed with a co-gellant system that includes a specific group of polyamide gelling agents previously described in (1) U.S. Pat. No. 6,051,216 (WO 99106473); (2) a case filed as Provisional application Ser. No. 60/229,445 on the same day as 60/229,444 (3) U.S. patent application Ser. No. 9/873,504 based on (2); and (4) a third application based on (2) and (3) being filed on the same date as this patent application as Attorney Docket Numner 6331-01 all of which are incorporated by reference herein in their entirety.

Antiperspirant products are well known in the art. Antiperspirant products have appeared in the marketplace in various dosage forms, such as sticks, gels, roll-ons, aerosols and creams. Generally, these dosage forms include a solution of the active ingredient in a solvent, a suspension of the active ingredient in a non-solvent, or a multi-phase dispersion or emulsion in which a solution of the active ingredient is dispersed in some continuous phase or in which the solubilized active ingredient constitutes a continuous phase.

Clear or translucent antiperspirant gels (which have been dispensed from containers having the appearance of a stick) have been marketed, consisting of viscous, high internal phase emulsions. These gels exhibit some advantages but these emulsions also suffer from various disadvantages, including often requiring the use of ethanol to achieve desired aesthetics. In connection with these emulsions, see U.S. Pat. No. 4,673,570 to Soldati and PCT (International Application) Publication No. WO 92/05767.

U.S. Pat. No. 5,120,531 to Wells, et al discloses rinse-off hair conditioner and styling compositions providing a gel-network thickened vehicle for the styling polymer and solvent. This patent discloses various siloxanes as the conditioning agent including polydiorganosiloxanes having quaternary ammonium-substituted groups attached to the silicon, and polydiorganosiloxanes having silicone-bonded substituents which are amino-substituted hydrocarbon groups.

U.S. Pat. No. 5,500,209 discloses a gel or stick which includes active deodorant and/or antiperspirant ingredients, a polyamide gelling agent, and a solvent for the polyamide gelling agent, in which the gel or stick composition can be clear or translucent. This patent discloses that the polyamide gelling agent is soluble in a cosmetically acceptable solvent at elevated temperatures, and solidifies (gels) upon cooling; acceptable solvents are disclosed as including various alcohols, including various glycols.

Addressing this problem of tackiness and stickiness in connection with cosmetic compositions utilizing a polyamide gelling agent, U.S. patent application Ser. No. 08/426,672, now U.S. Pat. No. 5,603,925, the contents of which are incorporated herein by reference in their entirety, discloses the use of a specific solvent system for a solid composition containing an antiperspirant active material and a polyamide gelling agent. This solvent system is glycol-free and contains a non-ionic surfactant and a polar solvent. Water is the polar solvent, and the non-ionic surfactant acts as a dispersing medium for the antiperspirant active material, in which sufficient water is used to give a clear or translucent solution/emulsion of the antiperspirant active material.

A typical technique to reduce the tackiness of, for example, antiperspirant formulations is the incorporation of one or more cyclomethicones (tetra-penta- or hexa-cyclodimethyl-siloxanes or mixtures thereof). These cyclomethicones are very low-viscosity silicone liquids that provide excellent lubricity but do not leave stains on the skin and/or clothing. More than 50% by weight of cyclomethicone has been incorporated into solid stick antiperspirant formulations, for example, using a wax solidifying agent. However, cyclomethicone is a nonsolvent for the dimer based polyamides described as gelling agents in U.S. Pat. No. 5,500,209. Moreover, only limited quantities of the cyclomethicone can be incorporated in solid compositions gelled using such polyamide gelling agent, without destroying the clarity of the gelled composition.

U.S. Pat. No. 5,243,010 to Choi, et al., discloses aromatic polyamide resins having pendant silyl groups.

U.S. Pat. No. 5,272,241 to Lucarelli, et al., discloses organofunctional siloxanes useful in both the personal care and plastics industries, the siloxanes being amino acid functionalized silicones.

U.S. Pat. No. 5,919,441, assigned to The Mennen Company describes in general the use of polyamides as gelling agents for cosmetic compositions.

PCT case WO 98/27951 assigned to Procter & Gamble discloses anhydrous, low residue gel-solid sticks having visible residue index of from 11-30 L-value which comprise a solid non-polymeric gellant this is substantially free of dibenzylidene alditol, inorganic thickening agents, organic polymeric gellants, n-acyl amino acid derivatives, or combinations thereof and which is also substantially free of selected polar solvents.

Gels, pastes and creams (which are also known as soft-solids or semi-solids) can be suitably packaged in containers which have the appearance of a stick, but which dispense through apertures (for example, slots or pores) on the top surface of the package. Reference is made to U.S. Pat. No. 5,102,656 to Kasat, No. 5,069,897 to Orr, and No. 4,937,069 to Shin, each of which discloses such gels, including physical characteristics thereof such as viscosity and hardness.

A representative composition which can be dispensed through apertures is described in U.S. Pat. No. 5,102,656 to Kasat. This disclosed composition is a creamy, heterogeneous anhydrous antiperspirant product containing, in percent by weight, of the total weight of the composition, 30%-70% of a volatile silicone as a carrier, 7-30% of a suitable gelling agent or agents, and about 12-30% of a physiologically acceptable antiperspirant agent. This patent discloses that the gelling agent can be any of a number of materials, including, for example, hydrogenated vegetable oil, hydrogenated castor oil, fatty acids having from 14 to 36 carbon atoms, beeswax, paraffin wax, fatty alcohols having from 14 to 24 carbon atoms, polyethylene and the like.

Other gellant systems that may be used include those made with an n-acyl amino acid such as N-lauroyl-glutamic acid derivative. Examples of such gelling systems include those described in U.S. Pat. Nos. 5,429,816; 5,733,534; 5,776,494; 5,591,424; 5,840,287; 5,843,407; 5,846,520; 5,849,276; 5,965,113; 6,190,673 and 6,241,976.

Notwithstanding the foregoing, there is still a need for providing antiperspirants/deodorants with improved gelling systems, especially when such systems may be used to obtain products exhibiting low white residue on both skin and fabric. It is also an overall object of the present invention to provide antiperspirants/deodorants comprising a co-gellant system wherein the products obtained exhibit good aesthetics as well as better dry glide-on feel.

### SUMMARY OF THE INVENTION

The emphasis of this invention is on products having low white residue on skin and fabric, particularly having a residue of less than 0.55 as measured by a reflectometer on human underarm skin (equipment such as the Chroma meter, CR-300 reflectometer (Minolta, Japan)). These compositions include products made as opaque suspensions and translucent to opaque emulsions (especially opaque emulsions). Opaque suspensions are of special interest. Low white residue antiperspirant/deodorant compositions of the invention can be formed comprising:

(a) a primary gellant of from 4-15% of a selected siliconized polyamide as described below;

(b) a secondary gellant (also called herein a co-gellant) which is up to 10% (for example up to 5%) of at least one member selected from the group consisting of N-acyl amino acid derivatives, particularly amides, (for example and particularly, dibutyl lauroyl glutamide (also referred to as N-lauroyl-glutamic acid amide (such as GP-1 from Ajinomoto)); dibenzylidene sorbitol (“DBS”); N,N′-hexamethylenebis-(10-undecenamide); amine stearate (for example, Kemamide W-40 from Witco, Greenwich, Conn.); 12-hydroxystearic acid; stearyl alcohol and waxes (for example, castor waxes);

(c) a solvent system for the primary and secondary gellants in an amount of up to 90%; and

(d) a high density antiperspirant active having a bulk density of at least 0.61 g/cm³ and used in an amount to have a deodorant and/or antiperspirant effect, wherein all the amounts are in percent by weight based on the total weight of the composition.

The siliconized polyamides useful in this invention are related to those described in U.S. Provisional application No. 60/229,445 and the follow-up cases filed that claim priority from this case as listed above; however the compositions in this current case are opaque suspensions or translucent to opaque emulsions as described below, while the compositions in the cases based on U.S. Provisional application No. 60/229,445 are clear emulsions.

The products of the invention can be made either as auspension or as an emulsion with dissolved active being an internal phase for the emulsion. While the products are either opaque or translucent in the package, upon application to the underarm they exhibit low white residue on both skin and black fabric. If the products are made as opaque suspensions, the antiperspirant active is added as a powder to the composition during manufacture. If the products are made as emulsions, the antiperspirant active is dissolved in a solvent such as water, one or more glycols (as defined below), or mixtures of water and glycol. If only water is used as the solvent, the amount is ≦25 weight %. If only glycols are used as the solvent or if a water/glycol mixture is used as the solvent, in each case the amount is ≦35 weight %. All amounts are based on the total weight of the composition.

The products can be made to form creams (for example, semi-solid or soft solid) and sticks; thus, both soft or firm compositions can be formed. The firmness of the product will depend on the amount of the gelling agent(s) used and the type and amount of emollients.

Optional ingredients such as emollients, silicone gums, elastomers, silicone resins, colorants, fragrances, surfactants, and inert particulates may be used to achieve better structural integrity or aesthetics.

### DETAILED DESCRIPTION OF THE DRAWINGS

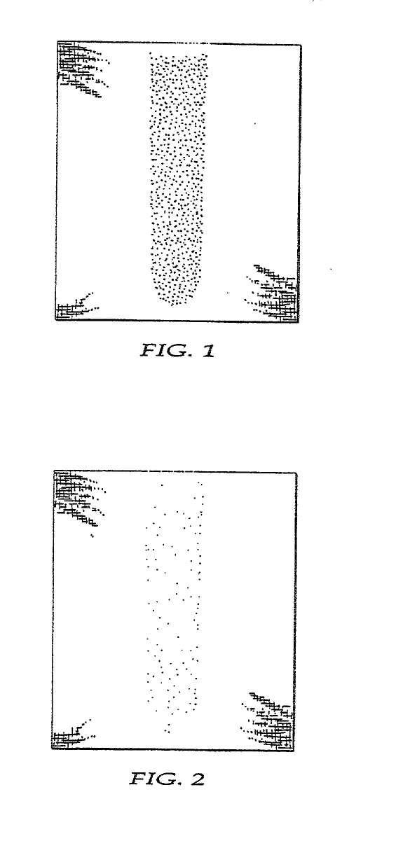

FIGS. 1 and 2 represents a comparison of residue from commercial stick products (FIG. 1) versus products of the invention (FIG. 2). The stippling shows the relative amount of residue left on black acetate cloth when a stick is wiped across the cloth once with a force of approximately 5 Newtons applied perpendicular to the cloth.

For FIG. 1, samples of SECRET Sheer Dry and Lady Speed Stick Invisible Dry were evaluated.

For FIG. 2, Examples 4 and 7 were evaluated. The stippling represents the relative amount of residue left on black acetate cloth.

### DETAILED DESCRIPTION OF THE INVENTION

This invention comprises antiperspirant and/or deodorant compositions exhibiting a low white residue on skin and black fabrics, which comprise:

(a) from 4-15% by weight based on the total weight of the composition of at least one siliconized polyamide of Formula IIIA as a first gellant:

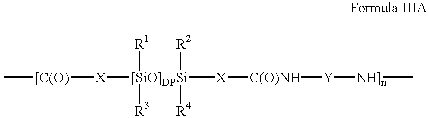

where:

(1) DP is a number in the range of 5-30, particularly 12-18 (more particularly 15);

(2) n is a number selected from the group consisting of 1-500 (particularly 20-200);

(3) X is a linear or branched chain alkylene having 1-30 carbons;

(4) Y is selected from the group consisting of linear and branched chain alkylenes having 1-40 carbons, wherein:

(A) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C1-C3 alkyls; and

(B) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y=Z where

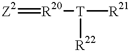

wherein each of R²⁰, R²¹ are independently selected from the group consisting of linear and branched C1-C10 alkylenes; R²² is selected from the group consisting of linear and branched C1-C10 alkanes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl, especially methyl and ethyl and most especially methyl; and

(5) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl (with more particular values for R¹-R⁴ being selected from methyl and ethyl and especially methyl;

wherein the polyamide of Formula IIIA has:

(i) a silicone portion in the acid side of the polyamide; and

(ii) an average molecular weight of at least 10,000, especially at least 30,000 daltons (for example, in the range of 90,000-120,000); and

(iii) a polydispersity of less than 20 (particularly less than 10 and, more particularly, less than 4);

(b) up to 10 weight % (particularly up to 5% and more particularly from 0.5-2.0%) of at least one co-gellant selected from the group consisting of N-acyl amino acid derivatives (for example, dibutyl lauroyl glutamide (also called N-lauroyl-L-glutamic acid di-n-butylamide or N-lauroyl-glutamic acid amide); dibenzylidene sorbitol (“DBS”); N,N′-hexamethylenebis-(10-undecenamide); amine stearate; 12-hydroxy stearic acid; stearyl alcohol, and waxes such has castor waxes (with a particular group of co-gellants being selected from the group consisting of N-acyl amino acid derivatives (for example, dibutyl lauroyl glutamide); dibenzylidene sorbitol (“DBS”); N,N′-hexamethylenebis-(10-undecenamide); and amine stearate; and a particular co-gellant being, dibutyl lauroyl glutamide, especially in an amount of 1.2-1.5 weight

(c) solvent system for the primary and secondary gellants in an amount of up to 90% wherein the solvent system is compatible with the primary gellant and co-gellant and the solvent system comprises one or more members is selected from the group consisting of:

(1) from 5-65% by weight (particularly 10-30%) based on the total weight of the composition of at least one non-silicone organic selected from the group consisting of C12-36 esters (for example, tridecyl neopentanoate, ethyl oleate, dioctyl carbonate, isopropyl myristate, octyl methoxycinnamate); guerbet alcohols having 8-30 carbons; fatty alcohols having 8-30 carbons (for example isostearyl alcohol and octydodecanol); ethoxylated and propoxylated alcohols having 3-30 carbons (for example, PPG-14 butyl ether, and PPG-3 myristyl ether such as in an amount of 24%); alkyl ethers having 12-36 carbons (for example, dioctyl ether); C12-18 alkyl benzoate and benzoate ester derivatives (for example, C12-15 alkyl benzoate, isostearyl benzoate and octyl dodecyl benzoate, octyl salicylate); and paraffins having a distillation temperature in the range of 372-426 degrees C.; isoparaffins having a distillation temperature in the range of 178-207 degrees C.; C6-30 alkyl carbonates (for example, dioctyl carbonate).

(2) from 2-55% by weight based on the total weight of the composition of a volatile silicone selected from the group consisting of cyclomethicones and low viscosity dimethicones (for example, Dow Coming 200 Fluid/2 centistokes or less from Dow Corning, Midland, Mich.);

(3) from 0-10% organo-silicones as described by Formula IA below (for example, phenyl trimethicone); and

(4) from 0-40% of a functionalized silicone as described in Formula V below. such as phenyl trimethicone;

(d) a high density antiperspirant active having a bulk density of at least 0.61 g/cm³ and used in an amount to have a deodorant and/or antiperspirant effect (for example Reheis AZP 908-0) from Reheis Incorporated, Berkeley Heights, N.J.); and Westchlor 30 BDM HBD (from Westwood Chemical Company, Middletown, N.Y.), wherein the amounts are in percent by weight based on the total weight of the composition.

The solvent system consists of one or more of the listed materials. The solvent system also allows the compositions of the invention to be processed at lower temperatures (for example, temperatures in the range of about 120-130 degrees C. or lower rather than temperatures in the range of 140-160 degrees C.). This is important in reducing the evaporation of volatiles from the composition during manufacturing and processing. It should also be noted that many of the solvents described have emollient characteristics in the overall formula.

While the high density antiperspirant active may be added to the composition as a dry powder or any antiperspirant active can be added as a premixed solution (for example, dissolved in water, propylene glycol, a mixture of water and propylene glycol or some combination of the glycols listed above either with or without water). It is preferred to add the antiperspirant active as a dry powder to obtain a product with better efficacy and aesthetics.

Optional ingredients (other than those described above) may also be added to the composition of the invention. These optional ingredients include additional emollients (0-20%), silicone gums (0-20%), elastomers (0-20%), silicone resins (0-20%), colorants (0-1%), fragrances (0-3%), antimicrobials (0-2%), surfactants (0-10%), and inert particulates (0-30%) to achieve better structural integrity, stability or aesthetics.

The basis of the invention is a co-gellant system made with one or more of the selected polyamides as described above, one or more of the secondary gellants as described above, an appropriate solvent system for the gallant(s) and co-gellant(s), and high density antiperspirant active(s). It has been found that the gellant system described herein gives low white residue products with good structural integrity and aesthetics. Not only is there low white residue on the skin, there is also low white residue on black fabric.

As noted above, the general class of polyamides from which further selections for the invention are made herein is the class generally described in copending case WO 99/06473 and the Attorney Docket Number IR 6331-01 case filed concurrently with this case. For the sake of clarity similar nomenclature is used here with the modifications as needed for the invention. This general description is followed by the particular description of the siliconized polyamides which give the superior results reported here. For the general description, these polyamides are multiples of a unit represented by Formula IIIA. The values for X, Y, DP, and R¹-R⁴ may be the same or different for each unit of the polyamide.

By siloxane groups is meant groups having siloxane units:

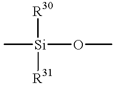

where R³⁰ and R³¹ are each independently selected from the group consisting of organic moieties, and each of R³⁰ and R³¹ are connected to the silicon by a carbon-silicon bond.

For the polyamides, the carbon numbers in the alkylene chain do not include the carbons in the extra segments or substitutions. Also, the polyamides must have a siloxane portion in the backbone and optionally may have a siloxane portion in a pendant or branched portion.

If repeated with no variations in the defined variables, Formula IIIA is representative of a linear homopolymer. Acceptable variations of the invention include: (1) polyamides in which multiple values of DP, X, Y, and R¹-R⁴ occur in one polymeric molecule, wherein the sequencing of these units may be alternating, random or block; (2) polyamides in which an organic triamine or higher amine such as tris(2-aminoethyl)amine replaces the organic diamine in part, to produce a branched or crosslinked molecule; and (3) physical blends of any of (1) and (2) and/or linear homopolymers.

Particular examples of compounds of Formula IIIA include the following:

1) Polyamides of Formula IIIA where the values for X, Y, n, and DP are the same as defined in Formula IIIA, and R¹-R⁴ are each methyl;

2) Polyamides of Formula IIIA where the DP is in the range of 5-30 (particularly 12-18 and more particularly 15);

3) Polyamides of Formula IIIB:

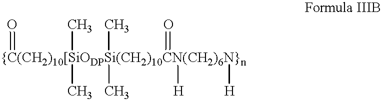

where DP is from 5-30 and n has the same value as in Formula IIIA;

4) Polyamides of Formula IIIB wherein the DP is from 5-20;

5) Polyamides of Formula IIIB wherein the DP is from 12-18;

6) Polyamides of Formula IIIB wherein the DP is 15;

7) Polyamides of Formula IIIA where the values of X, Y, DP and R¹-R⁴ remain the same in each unit of the polymer;

8) Polyamides of Formula IIIB where the value of DP and n remain the same for each unit of the polymer;

9) Polyamides of Formula IIIA containing multiple siloxane block lengths as shown in Formula IIIC:

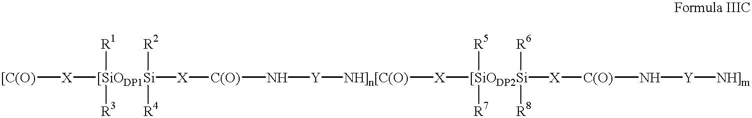

where X, Y, n, and R¹-R⁴ have the meanings described above for Formula IIIA; m is selected from the same groups as defined for n, and n and m denote the total number of units enclosed within the brackets, with the individual units arranged with regular, alternating, block or random sequencing; R⁵-R⁸ is selected from the same group as defined for R¹-R⁴; DP1 and DP2 may be the same or different and are each independently selected from the same group as defined for DP; and the units denominated by n and m may be structured to form either block (regularly sequenced) or random copolymers.

10) Polyamides of Formula A containing siloxane block lengths of Formula IIIC wherein all of the R groups are selected to be methyl.

11) Polyamides of Formula IIIA containing siloxane block lengths of Formula IIIC wherein DP1=DP2.

12) Polyamides of Formula IIIA containing siloxane block lengths of Formula IIIC wherein all of the R groups are selected to be methyl and DP1=DP2.

13) Polyamides synthesized from multiple diamines as shown in Formula IIID:

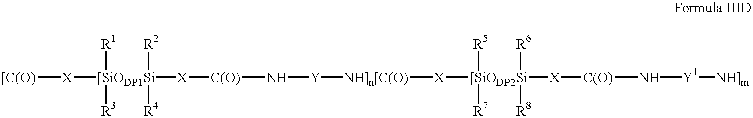

where X, Y, m, n, and R¹-R⁸, DP1, DP2 have the same meanings as described above for Formula IIIA and Formula IIIC; Y¹ is independently selected from the same group as defined for Y; and the units denominated by n and m may be structured to form either block (regularly sequenced) or random copolymers.

14) Polyamides of Formula IIID where DP1=DP2.

15) Polyamides of Formula IIID where all of the R groups are selected to be methyl.

16) Polyamides of Formula IIID where all of the R groups are selected to be methyl and DP1=DP2.

Another related class of polyamides may be synthesized with trifunctional amines as shown in Formula IV:

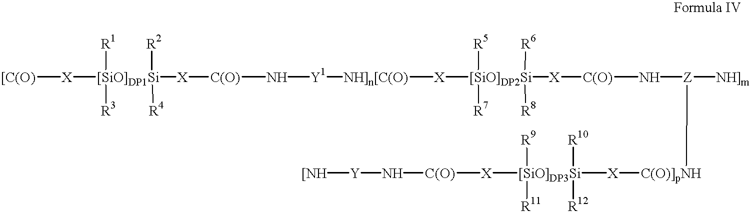

where X, Y, Y¹, R¹-R⁸, m, n, DP1-DP2, have the same values as defined above; R⁹-R¹² are selected from the same group as defined for R¹-R⁸, DP3 is selected from the same group as defined for DP; and p is selected from the same groups as defined for m and n;

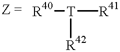

wherein R⁴⁰, R⁴¹ and R⁴² are each independently selected from the group consisting of linear and branched C1-C10 alkylenes, and T is selected from the group consisting of (1) and a trivalent atom selected from N, P and Al; and (2) CR, where R is selected from hydrogen and the same group as defined for R¹-R⁴. Preferred values for p are 1-25 with more preferred values being 1-7. Preferred values for R¹-R¹² are methyl. A preferred value for T is N. Particular values for each of DP1-DP3 are 5-30, particularly 5-20, more particularly 12-18 and especially 15. A preferred value for each of R⁴⁰, R⁴¹ and R⁴² is ethylene. A preferred value for Z=(—CH₂CH₂)₃N.

A particular group of compounds of Formula IV are those of Formula IVA:

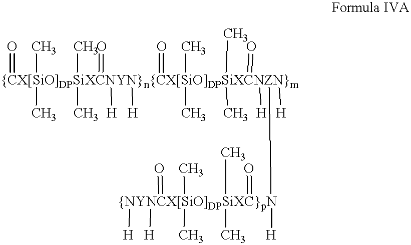

where X=—(CH₂)₁₀—, Y=—(CH₂)₆—; DP=15-45; and Z=(—CH₂CH₂)₃N; m=2-500 (particularly 20-200); n=2-500 (particularly 20-200); p=2-500 (particularly 20-200); provided m=5-20% of m+n+p and m, n, and p are selected so that the average molecular weight is at least 10,000 daltons and, preferably, at least 30,000 daltons.

In general, the siloxane-based polyamides (1) contain both siloxane groups and amide groups to thicken compositions containing silicone fluids (volatile and/or non-volatile silicone fluids); (2) are non-flowable solids at room temperature; and (3) dissolve in a fluid which contains silicone at a temperature of 25-160 degrees C. to form an opaque, translucent or clear product at a temperature in this range.

With regard to the siloxane units in the siloxane-based polyamides, the siloxane units must be in the main or backbone chain but can also optionally be present in branched or pendent chains. In the main chain the siloxane units occur in segments as described above. In the branched or pendent chains the siloxane units can occur individually or in segments.

Particular groups of siloxane-based polyamides include:

(a) polyamides of Formula IIIA where DP is a number in the range of 5-30, particularly 15-20, more particularly 12-18 and especially 15, provided that at least 8% of the composition is a polyamide of Formula IIIA with a DP in the range of 12-18, especially 15;

(b) physical blends of two or more polyamides described above in Formulae IIIA, IIIB, IIIC, IIID, IV and IVA, wherein (1) at least 80% of the blend is at least one polyamide as described above for this invention with a DP in the range of 5-30 with at least 8% of the final cosmetic composition being a polyamide of Formula IIIA with a DP in the range of 12-18, especially 15; and (2) the remainder of the blend is a polyamide of the Formulae IIIB, IIIC, IIID, IV, or IVA, except that the DP value is a number in the range of 45-500, or blends of these higher DP materials;

(c) compounds of Formula IIIC where (1) the value for DP1=5-30 and the value for DP2=5-500 and (2) the portion of the polyamide having DP1 is about 1-99 weight % based on the weight of the total polyamide content and the portion of the polyamide having DP2 is about 1-99 weight % with at least 8% of the final cosmetic composition being a polyamide of Formula IIIC with a DP in the range of 12-18, especially 15;

(d) physical blends of polyamides of Formula IIIB made by combining (1) 60-99 weight % of a polyamide where DP=5-30 and especially where DP=10-20, and (2) 1-20 weight % of a polyamide where DP=5-500, especially where DP=45-100 with at least 4% of the final cosmetic composition being a polyamide of Formula IIIB with a DP in the range of 12-18, especially 15;

(e) polyamides of Formula IIID where at least one of Y and Y¹ contains at least one hydroxyl substitution with at least 8% of the final cosmetic composition being a polyamide of Formula IIID with a DP in the range of 12-18, especially 15;

(f) polyamides of Formula IIIA synthesized with at least a portion of an activated diacid (diacid chloride, dianhydride or diester) instead of the diacid, with at least 8% of the final cosmetic composition being a polyamide of Formula IIIA with a DP in the range of 12-18, especially 15;

(g) polyamides of Formula IIIA where X=—(CH₂)₃— with at least 8% of the final cosmetic composition being a polyamide of Formula IIIA with a DP in the range of 12-18, especially 15;

(h) polyamides of Formula IIIA where X=—(CH₂)₁₀— with at least 8% of the final cosmetic composition being a polyamide of Formula IIIA with a DP in the range of 12-18, especially 15;

(i) polyamides of Formula IIIA where the polyamides are made with a monofunctional chain stopper selected from the group consisting of monofunctional amines, monofunctional acids, monofunctional alcohols, including fatty acids, fatty alcohols and fatty amines, such as, for example: octylamine, octanol, stearic acid and stearyl alcohol with at least 8% of the final cosmetic composition being a polyamide of Formula IIIA with a DP in the range of 12-18, especially 15.

In general, an amount of polyamide equal to at least 4% by weight based on the final weight of the total antiperspirant and/or deodorant product should be used. This is especially true if a polyamide of Formula IIIA having a DP=15 is used. If a polyamide with a DP=30 is used, about 5-15% more polyamide must be used for stick products.

It should also be noted that nomenclature is being developed to call this type of polyamide “nylon/dimethicone copolymers” such as “nylon 611/dimethicone copolymer”, where “611” means that the organic portion of the copolymer has 6 and 11 carbons on either side of the amide group.

While one method for making polyamides is described in U.S. Pat. No. 6,051,216 listed above, another method for making such polyamides is described in U.S. Pat. No. 5,981,680, both of which are incorporated by reference as to the methods of making such compositions. The process of U.S. Pat. No. 5,981,680 involves the addition of an olefinic acid with an organic diamine to product an organic diamide. Once the olefinic acid and the organic diamine are fully reacted, an ≡SiH endblocked polysiloxane is added in the presence of a platinum catalyst to product a siloxane-based polyamide via hydrosilylation.

As noted above, the two major factors in describing the polyamides of this invention are DP and molecular weight. Optimal polymers are formed from the reaction of a siloxane diacid with a DP=5-30, more particularly 12-18, and especially 15, and an organic polyfunctional amine (for example, hexamethylenediamine). (Note that the five-step method uses a siloxane diacid with a diamine and a three-step method (see U.S. Pat. No. 5,981,680) use siloxane plus diamide.) Polymers having molecular weights (“M_{W}”) in the range of 4,000-200,000 may be produced, especially those in the range of 50,000-150,000. Reference is made to U.S. Pat. No. 6,051,216 and U.S. patent application Ser. No. 9/873,504 described above for methods that may be used to obtain such polymers. Reference is also made to a U.S. patent application filed on Jul. 12, 2001, by Dow Coming Corporation as their Docket Number DC4882 which is incorporated by reference herein as to its method of making selected polyamides. This most recent case uses a siloxane and diamide method which is improved by the attention directed to chain terminators and reactant ratios. It is believed that this most recent case, at the very least, describes a commercially more efficient way of producing polyamides described for this invention, especially in the range of 80,000-150,000 daltons, particularly 80,000-120,000 daltons (with a particular example being 80,000-90,000 daltons), and more particularly 90,000-120,000 daltons.

Polyamides having a molecular weight in the range of 90,000-120,000 daltons and a degree of polymerization in the range of 12-18, especially 15, are especially useful in practicing the invention, however, clarity does not have to be maintained so other ingredients may be used which are opacifying.

Optimizing the length of the siloxane portions of the molecule (the “DP”) involves a balancing of various considerations. Polyamides with long siloxane chains (for example, DP>50) tend to produce soft gels in cyclomethicone. The efficiency of the polyamide gellant is improved by reducing the length of the siloxane units (that is, selecting and making a molecule with a DP<50), but the compatibility with cyclomethicone may be compromised as the DP decreases. For example, a polyamide synthesized from a siloxane diacid with a DP=15 and hexamethylenediamine does not produce clear gels in cyclomethicone. As a result, polymers with DP=15 are preferred, so that the formulation for the resulting cosmetic composition has a combination of some compatibility with silicone fluids and good gelling efficiency. It should be noted that frequently more than one emollient is normally used to achieve the preferred aesthetics, for example, with a DP=15.

In addition to the DP of the polyamide, the molecular weight must also, be considered. Polymers of extremely high molecular weight (for example, greater than 200,000 daltons) tend to produce rubbery, elastic gels and are less desirable. It has been found that optimal gelation occurs with polyamide gellants of molecular weight greater than 70,000 as determined by size exclusion chromatography with universal calibration as described in Styring, J. E. et al “An Experimental Evaluation of a New Commercial Viscometric Detector for Size-Exclusion Chromatography (SEC) Using Linear and Branched Polymers,” J. Liquid Chromatography, Volume 9, pages 783-804 (1986). In practicing the current invention, the optimal range of molecular weights for the primary gellant should be from 50,000-150,000 daltons, especially 70,000-120,000 daltons (with a particular example being 80,000-90,000 daltons), and more especially 90,000-120,000 daltons. It is believed, however, that incorporation of low levels of such high molecular weight species, for example, 0.5 weight % of a high molecular weight polyamide having a molecular weight in the range of 120,000-200,000 may give the base composition and cosmetic compositions made therefrom improved mechanical properties.

It has been found that selecting siliconized polyamides with certain values for polydispersity and suitable stress/strain properties has an important affect on being able to form stick products. Polydispersity is calculated as M_{W}/M_{N} where M_{N} is number average molecular weight and M_{W} is weight average molecular weight. More particularly, when the molecular weight of the siliconized polyamide is increased while the polydispersity of the polyamide gellant remains narrow, the strength of the formulated product increases. The strength of the formulated product is monitored using a Three Point Bending technique as found in An Introduction to the Mechanics of Solids, (edited by Lardner, T. J.; McGraw-Hill 1978). A failure stress greater than 2.0 Pascals (and preferably greater than 4.0 Pascals) is desired for a stick product. If the failure stress is less than 2.0 Pascals, a softer stick can be formed.

As noted above, the siloxane-based polyamides used as one of the co-gellants in this invention contain both siloxane units and amide linkages. The siloxane units provide compatibility with the silicone fluid (for example with the cyclomethicones), while the amide linkages and the spacing and selection of the locations of the amide linkages facilitate gelation and the formation of cosmetic products.

With respect to the co-gellants described in section (b), up to 10% (particularly up to 5%) can be used, with 0.5-2.0% being a preferred amount. These secondary gellants are preferably, but not limited to, non-polymeric materials whose gelatin mechanism is through crystalline network. This type of gellant is described by Terech and Weiss in Chemical Review, 1997, 97, 3133-3159.

The solvent system is a critical element for this invention, because it dissolves the gellant and co-gellant at a desired elevated temperature and forms a cohesive gel upon cooling. The dissolution temperatures of the gellant and co-gellant are in the range of 60 to 200° C., preferably 80 to 160° C., and most preferably 90-120° C. As mentioned earlier, these solvents could include one or more members selected from the groups consisting of:

(1) non-silicone organics;

(2) volatile silicones;

(3) organo-silicones; and

(4) functionalized silicones.

Preferably, the volatile silicone fluid includes cyclomethicones. The cyclomethicone used (that is, ring size of the cyclomethicone) has an effect on the hardness of the gels formed. That is, cyclomethicone having five siloxane units produces a softer gel than that produced utilizing a material with 6 siloxane units. As the ring size of the cyclomethicone increases, the rigidity of the gel system formed increases. As described above, particular examples of suitable cyclomethicones include those having rings of 4-6 siloxane units, especially “D5”.

The volatile low viscosity methylsilicone fluid contains dimethylsiloxane units and, optionally, trimethylsiloxane units. Representative compounds are cyclopolysiloxanes of the formula [(CH₃)₂SiO]ₓ, and linear short chain siloxane compounds of the formula (CH₃)₃SiO[(CH₃)₂SiO]_{y}Si(CH₃)₃ in which x is an integer having a value of from three to ten, (especially 4-6) and y is an integer having a value of from zero to about four. The cyclopolysiloxanes have been assigned the INCI name “CYCLOMETHICONE” by The Cosmetics, Toiletries and Fragrance Association, Inc., Washington, D.C. (CTFA).

For organosilicone fluids, the cosmetic compositions can include from 0-10%of an organosilicone that is selected from Formula IA (or mixtures thereof):

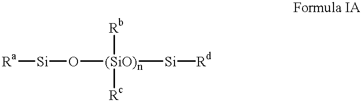

wherein each of R^{a}, R^{b}, R^{c}, and R^{d} may be the same or different and are each independently selected from the group consisting of hydrogen, C1-C15 alkyl (for example, methyl, ethyl, propyl, isopropyl), phenyl, and C1-C15 alkyl itself containing a member selected from the group consisting of, —OH, —COOH, —NH₃ —CO(O)—, and n is a number in the range of 5-500. Examples of compositions of Formula IA include phenyltrimethicone, caprylyl methicone, and phenethyl dimethicone.

Throughout the present specification, “antiperspirant active” and “deodorant active” materials are discussed. Both types of materials contribute to reduction of body malodor, for example, axillary malodor. By reduction of body malodor, it is meant that, generally, there is less body malodor after application of the composition to a person's skin, as compared to a person's malodor without application of the composition. Such reduction can be due to a masking of the malodor, absorption and/or chemical reaction of the malodorous material, reduction of the levels of the bacteria producing the malodorous materials, for example, from perspiration, reduction of perspiration, etc. The antiperspirant active materials, when utilized in appropriate amounts, primarily act to reduce malodor by reducing perspiration; the antiperspirant active materials can also have a deodorant function, for example, as an antimicrobial or bacteriostatic agent. The deodorant active materials do not substantially reduce perspiration, but reduce malodor in other ways. For example, as fragrances masking the malodor or reducing the malodor intensity; absorbents; antimicrobial (bacteriostatic) agents; or agents chemically reacting with malodorous materials.

Where the composition contains an antiperspirant active, any of the known antiperspirant active materials can be utilized provided they meet the required minimum bulk density. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex PG, aluminum chlorohydrex PEG, aluminum dichlorohydrex PG, and aluminum dichlorohydrex PEG. The aluminum-containing materials can be commonly referred to as antiperspirant active aluminum salts. Generally, the foregoing metal antiperspirant active materials are antiperspirant active metal salts. In the embodiments which are antiperspirant compositions according to the present invention, such compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as aluminum nitratohydrate and its combination with zirconyl hydroxychlorides and nitrates, or aluminum-stannous chlorohydrates, can be incorporated as an antiperspirant active ingredient in antiperspirant compositions according to the present invention.

Antiperspirant actives can be incorporated into compositions according to the present invention in amounts in the range of 0.1-25%, 5-25 percent, and preferably 15-25%, by weight, of the total weight of the composition. The amount used will depend on the formulation of the composition. For example, at amounts in the lower end of the broader range (for example, 0.1-10%), the antiperspirant active material will not substantially reduce the flow of perspiration, but will reduce malodor, for example, by acting as an antimicrobial material.

The bulk density of the antiperspirant active (preferably of the aluminum zirconium type) must be at least greater than 0.45 g/cm³, and preferably greater than 0.61 g/cm³. It should be noted that the theoretical upper limit on bulk density is about 2.42 for aluminum chlorohydrate antiperspirant actives and about 4 for aluminum zirconium actives. It should also be noted that particles having a size of greater than 100 microns will be perceived by the consumer as scratchy. This would be undesirable from an aesthetic viewpoint.

If emulsions are formed, the composition can also include a solvent for the antiperspirant active. This solvent, which is not miscible with the silicone fluid, can illustratively be water, a glycol or polyglycol such as propylene glycol, dipropylene glycol, tripropylene glycol, butylene glycol, 1,2-hexanediol; dimethyl isosorbide; polyhydric alcohols having 3-9 carbons; polymeric ethers having 5-30 units selected from the group consisting of ethylene oxide and propylene oxide. The glycol or polyglycol is selected from the group consisting of ethylene glycol, propylene glycol, 1,2-propanediol, diethylene glycol, triethylene glycol, tetraethylene glycol, dipropylene glycol, tripropylene glycol, methyl propanediol, 1,6-hexanediol, 1,3-butanediol, 1,4-butanediol, PEG-4 through PEG-100, PPG-9 through PPG-34, pentylene glycol, neopentyl glycol, trimethylpropanediol, 1,4-cyclohexanedimethanol, 2,2-dimethyl-1,3-propanediol, 2,2,4,4-tetramethyl-1,3-cyclobutanediol, and mixtures thereof. More particular examples of the glycol component include one or more members of the group consisting of propylene glycol, dipropylene glycol, tripropylene glycol, 2-methyl-1,3-propanediol, methyl propylene glycol, low molecular weight (less than 600) polyethylene glycol, low molecular weight (less than 600) polypropylene glycols, and mixtures of any of the foregoing. Propylene glycol is of particular interest because the antiperspirant active is more soluble in this type of glycol. Tripropylene glycol has lower irritation, but the antiperspirant active is not as soluble in this glycol. Mixtures of glycols may be used to balance these desirable properties.

Optional ingredients include phenyl trimethicone as well as suitable functionalized silicone fluids are hydroxy functional fluids with the general structure of Formula V:

(R¹—Si—O_{3/2})ₐ—(R²₂—Si—O_{2/2})_{b}—((HO)R³₂—Si—O_{1/2})_{c} Formula V

where each of R¹, R², and R³, may be alike or different and are each independently selected from the group consisting of C1-C4 straight chain alkyls (especially methyl);

a is a number in the range of 0-10, with particular values of “a” being 0 for linear compounds and 1-10 for branched compounds (for example 6-8);

b is a number in the range of 0-10,000, with particular values of “b” being 4-6000;

c is a number in the range of 1-10, with particular values of “c” being 2 when the compound is linear and at least 3 when there is branching; provided that a and b cannot both equal zero at the same time. It is to be recognized that a, b, and c are average values (including whole numbers and fractions) and mixtures of compounds with various values for a, b, c, R¹, R², and R³ may also be used.

A particular group of these optional ingredients includes phenyl trimethicone and the following examples of compounds of Formula V:

(a) linear polydimethylsiloxanediols where a=0, b=4-6,000 (for example, an average value of 4, 40 or 6,000);

(b) linear polydimethylsiloxanediols where a=0, b=4-1,000 and c=2;

(c) multifunctional branched siloxanes where a=1-2, b=0-1,000, and c=3-4;

(d) linear polydimethylsiloxanediols where a=0, b=40 and c=2;

(e) multifunctional branched siloxanes where a=1, b=16, and c=3;

(f) multifunctional branched siloxanes where a=1-2, b=10-1,000, and c=3-4;

(g) mixtures of the particular compounds described in parts (a)-(f), for example, mixtures wherein the average structure of the mixture is described by a=0.1, b=4-6000, and c=2-7; and

(h) two component mixtures of the particular compounds described in parts (a) -(f) wherein one component is 0.1-99.9% of the composition and the other component is the remainder to 100%.

For each of the groups listed as (a)-(f) above, particular examples of the compounds are when each of the R groups is selected to be methyl. Also, for any of the groups (a)-(g), additional silicone fluids such as dimethicone may be added, for example in amounts of 0.1-90% functionalized silicone and 10-99.9% silicone fluid or fluids.

One particular group of compounds of Formula V are linear silanols of Formula VA, especially when b=40:

HO—(R³)₂Si—O—((R²)₂Si—O)_{b}—Si—(R³)₂OH Formula VA

Some of the compounds of Formula V may be purchased commercially. For methods of making other compounds of this invention descriptions of suitable methods may be found in the literature for example, U.S. Pat. No. 5,302,382 to Dow Corning; U.S. Pat. No. 3,441,537 to Stauffer Chemical Company; and Noll, W., Chemistry and Technology of Silicones, (Academic Press, Inc. Orlando, Fla. 1968) especially at pages 190-196 and 239-245, all of which are incorporated herein by reference to the extent they describe how to make these compounds.

While the hydroxy functionalized silicones described above are preferably selected to have a viscosity that does not require additional silicone materials (for example, having a viscosity in the range of up to 60,000 centistoke (cst), it is possible to use compositions which are a blend of hydroxy functionalized silicones having higher viscosities such as those having a high viscosity (>500,000 centistoke) dimethiconol in dimethicone where the dimethicone has a viscosity in the range of 5-350 centistoke (for example, DOW CORNING® 1403 Fluid).

For high viscosity functionalized silicones (for example, the silicone gums), and for the purpose of facilitating its handling and processing, these materials are generally provided as blends with another volatile or non-volatile low viscosity silicone such as CYCLOMETHICONE, or a non-volatile linear silicone fluid having a viscosity of about 5 to 350 centistoke. Such dimethyl silicone polymers terminated with hydroxyl groups have been assigned the INCI name “DIMETHICONOL” by The Cosmetics, Toiletries and Fragrance Association, Inc., Washington, D.C. (CTFA). Blends of such silicone gums with a volatile low viscosity cyclic silicone have been assigned the INCI name “CYCLOMETHICONE (and) DIMETHICONOL” by the CTFA. Other blends of such silicone gums with a non-volatile low viscosity linear silicone have been assigned the INCI name “DIMETHICONE (and) DIMETHICONOL” by the CTFA. The DIMETHICONOL content of such blends is typically in the range of about 12 to 14 percent by weight, and the blend viscosity may range from 500 to about 20,000 centistoke, generally in the range of about 4,000 to 5,000 centistoke. DIMETHICONE concentrations in the range of 10-48% are known or may be made from other concentrations.

Other volatile low viscosity methylsilicone fluids are described in U.S. Pat. No. 5,302,382 to Kasprzak, incorporated by reference herein. Examples of methylsilicone fluids having viscosities of less than about one hundred centistoke measured at twenty-five degrees Centigrade, preferably less than about five centistoke and also methylsilicone fluids having a viscosity in the range of 1-350 centistoke are disclosed.

The silicone fluid component can also, optionally, include other silicone materials even when the purpose is for reasons other than viscosity modification. Particular silicone fluids are selected so that a stable silicone/glycol suspension is formed when the two phases are combined and mixed. Such materials can include, for example, other silicone fluids such as polydimethylsiloxanes, polydiethylsiloxanes, and polymethylethylsiloxanes, having a viscosity in excess of 350 centistoke and up to 2,500,000 centistoke, preferably, 350-10,000 centistoke. Further examples include cetyl dimethicone copolyol, dimethicone copolyol (such as DOW CORNING® 2501, Q2-5220 and 5324 products); a mixture of cyclomethicone and dimethiconol (such as DOW CORNING® 1401 product); a mixture of dimethicone and dimethiconol (such as DOW CORNING® 1403 product); cetyl dimethicone (DOW CORNING® 2502 product); and stearyl dimethicone (DOW CORNING® 2503 product).

If an elastomer is used it will include at least one crosslinked organopolysiloxane material as a gelling agent and a vehicle as described herein. Suitable organopolysiloxanes are made from a cross-linking agent and at least one member selected from the group consisting of siloxanes containing at least one vinyl group (hereinafter referred to as a “vinyl polysiloxane”) and alpha, omega dienes. Suitable vinyl polysiloxanes include:

(a) vinyl terminated polysiloxanes such as that of Formula IE:

CH₂═CH—Si(CH₃)₂—O—(—Si(CH₃)₂—O—)ₙ—Si(CH₃)₂—CH═CH₂ Formula IE

(b) vinyl functional copolymers such as that of Formula IIE:

(CH₃)₃Si—O—(Si(CH₃)₂—O—)ₘ—(Si(CH₃)(CH═CH₂))ₙ—Si(CH₃)₃ Formula IIE

where n a number from 1-100, particularly 10-50; and m=a number from 1-100, particularly 10-50.

Particular examples of vinyl polysiloxanes include, but are not limited to:

(a) polydimethylsiloxane, which is monovinyl terminated;

(b) vinylmethyl, dimethylsiloxane copolymer which is trimethylsiloxy terminated;

(c) vinylmethyl, dimethylsiloxane copolymer which is vinyl dimethyl terminated;

(d) divinylmethyl terminated polydimethyl siloxanes;

(e) vinyl Q-resin

(f) vinylphenylmethyl terminated dimethyl siloxanes;

(g) cyclic vinylmethyl dimethyl siloxanes;

(h) T-structure polydimethyl siloxanes with vinyl at branchpoint;

(i) T-structure polydimethyl siloxane with vinyl at branch terminus;

(j) diphenyl dimethyl copolymer which is vinyl terminated;

(k) vinyl terminated polydimethyl siloxanes;

(l) vinyl terminated trifluoropropyl methyl siloxane—dimethylsiloxane copolymer;

(m) vinyl terminated diethyl siloxane copolymer;

(n) vinyl methyl siloxane—dimethyl siloxane copolymer which is trimethylsiloxy terminated

(o) vinyl gums;

(p) vinyl methyl siloxane homopolymers; and

(q) mixtures of two or more of the foregoing.

Suitable alpha, omega dienes include those described in U.S. Pat. No. 5,880,210 (incorporated by reference in its entirety herein), especially those of Formula: CH₂═CH(CH₂)ₓCH═CH₂, where x is a number in the range of 1-20. Particular examples of suitable alpha, omega dienes include: 1,4-pentadiene; 1,5-hexadiene; 1,6-heptadiene; 1,7-octadiene; 1,8-nonadiene; 1,11-dodecadiene; 1,13-tetradecadiene; and 1,19-eicosadiene.

Suitable crosslinking agents include hydride terminated polydimethylsiloxanes of Formula IIIE:

H—Si(CH₃)₂—O—(Si(CH₃)₂—O—)ₚ—Si(CH₃)₂—H Formula IIIE

where p=a number from 1-50, particularly 5-20.

Particular examples of cross-linking agents are hydride functional polymers (≡SiH). Typical hybrid crosslinking agents are methylhydro-dimethylsiloxane copolymer with 20-60% methyl hydrogen. In selected circumstances hydride terminated siloxanes may be used for chain extension. Examples of suitable crosslinking agents include but are not limited to:

(a) methylhydrosiloxane—dimethylsiloxane copolymer;

(b) polymethylhydrosiloxanes;

(c) polyethylhydrosilane;

(d) polyphenyl—(dimethylhydrosiloxy)siloxane which is hydride terminated;

(e) methylhydrosiloxane—phenylmethylsiloxane copolymer which is hydride terminated; and

(f) methylhydrosiloxane—octylmethylsiloxane copolymer.

The formation of such elastomer products is described in the references listed herein such as in U.S. Pat. No. 5,654,362 to Schultz, Jr. et al (incorporated by reference in its entirety herein).

Particular examples of suitable elastomers are SFE 167, a cetearyl dimethicone/vinyl dimethicone crosspolymer from GE Silicones (Waterford, N.Y.); SFE168, a cyclomethicone (and) dimethicone/vinyl dimethicone crosspolymer from GE Silicones; vinyl dimethicone crosspolymers such as those available from Shin Etsu Silicones of America (Akron, Ohio) under trade names KSG-15 (cyclomethicone (and) dimethicone/vinyl dimethicone crosspolymer), KSG-16 (dimethicone (and) dimethicone/vinyl dimethicone crosspolymer), KSG-17 (cyclomethicone (and) dimethicone/vinyl dimethicone crosspolymer), KSG-18 (phenyl trimethicone (and) dimethicone/phenyl vinyl dimethicone crosspolymer); and KSG-20 (dimethicone copolyol crosspolymer; dimethicone/vinyl dimethicone crosspolymer from Dow Coming Corporation (Midland, Mich.) under trade name Dow Coming 9506 Cosmetic Powder; and a mixture of cyclomethicone and stearyl-vinyl/hydromethylsiloxane copolymer available from Grant Industries, Inc. (Elmwood Park, N.J.) under the trade name Gransil SR-CYC.

Compositions according to the present invention can include other cosmetic additives conventionally incorporated in cosmetic compositions, including (but not limited to) perfumes, cosmetic powders, colorants, emulsifiers, emollients, waxes, organosilicones, fatty esters, fatty alcohols, bees wax, behenoxy dimethicone, stearyl alcohol, etc. and other cosmetic agents. As for various other ingredients which can be incorporated, attention is directed to the optional components such as colorants, perfumes and additives described in the following U.S. Patents: U.S. Pat. No. 5,019,375 to Tanner, et al (the contents of which are incorporated herein by reference in their entirety); U.S. Pat. No. 4,937,069 to Shin (the contents of which are incorporated herein by reference in their entirety); and U.S. Pat. No. 5,102,656 to Kasat (the contents of which have been previously been incorporated herein by reference in their entirety). The use of optional additives may, of course, adversely affect clarity.

Cosmetic compositions according to the present invention can also include surface active agents. For example, where the composition is an antiperspirant composition, containing antiperspirant active material, the antiperspirant active material can be included in the composition in a solution in, for example, water, and/or propylene glycol, which may not be miscible with the silicone fluid, and the composition can also include surface active agents so as to disperse the solution of antiperspirant active material in the composition.

It is preferred that some type of surface active agent be included in the final product to aid in the removal of product during bathing or showering. Suitable agents include, but are not limited to, ethoxylated carboxylic acids (for example, the polyethylene glycol diester of lauric acid that conforms generally to the formula CH₃(CH₂)₁₀C(O)—(OCH₂CH₂)ₙO—C(O)(CH₂)₁₀CH₃ where n has an average value of 8 (also called PEG-8 dilaurate); the polyethylene glycol diester of stearic acid that conforms generally to the formula CH₃(CH₂)₁₆C(O)—(OCH₂CH₂)ₙO—C(O)(CH₂)₁₆CH₃ where n has an average value of 8 (also called PEG-8 stearate)); ethoxylated glycerides (for example, a polyethylene glycol derivative of Castor Oil with an average of 4 moles of ethylene oxide (also called PEG-4 castor oil)); glycol esters (for example, propylene glycol ricinoleate); monoglycerides (for example, glycerol myristate); polyglyceryl esters (for example, polyglyceryl-4 oleyl ether); polyhydric alcohol esters and ethers (for example, sucrose distearate); sorbitan/sorbitan esters (for example, sorbitan sesquiisostearate); ethoxylated alcohols (for example, laureth-4); ethoxylated polysiloxanes (for example, dimethicone copolyol); propoxylated polyoxyethylene ethers (for example, the polyoxypropylene, polyoxyethylene ether of cetyl alcohol that conforms generally to the formula CH₃(CH₂)₁₄CH₂(OCH(CH₃)CH₂)ₓ(OCH₂CH₂)_{y}OH where x has an average value of 5 and y has an average value of 20 (also called PPG-5 ceteth-20)).

Additives may be added to the base composition to help add and incorporate active ingredients, improve mechanical properties, improve aesthetic properties, make a clear product, make a product with color, etc. Thus, cosmetic compositions may then be made by combining the base composition with one or more additional components, active ingredients, one or more vehicles to allow the active ingredient to combine more easily (or with more desirable properties) with the base composition, and other ingredients used by those in the art to formulate cosmetically acceptable products including fragrances, emollients, antibacterials hardeners, strengtheners, chelating agents, colorants, emulsifiers and other additives such as, silicas, silica-based resins, corn starch, alumina, fumed silica, calcium carbonate, clay, talc, high molecular weight polymers (for example silicone gums, elastomers).

As indicated previously, the compositions according to the present invention are sticks with varying degrees of rigidity depending on amounts of thickening agent incorporated in the composition as well as soft solids. It is difficult to quantitatively distinguish between a cosmetic “gel” and a cosmetic “stick”. Generally, a gel is more viscous than a liquid or than a paste which fails to retain its shape; however, it is not as rigid as a stick. Typically, it is understood that gels are soft, deformable products while sticks are free-standing solids. For example, by rheological analysis, a commercial deodorant stick has been determined to have a plateau storage modulus G′(ω) of roughly at least 10⁵ Pa and a complex viscosity of at least 10⁶ Pa second (both at an angular frequency of 0.1 rad-sec). On the other hand, a commercial antiperspirant gel or cream may have a G′(ω) value of roughly about 10²-10⁵ Pa and a complex viscosity in the range of about 10³-10⁶ Pa second (at 0.1 rad-sec).

Base and cosmetic compositions according to the present invention can easily be manufactured by methods known to those skilled in the art such as by using known mixing procedures. Base compositions according to the present invention can be made by mixing the various components at an elevated temperature (that is, by heating and mixing the various components) and then cooling in order to form the gelled (solidified) stick composition. For cosmetic compositions, the additional ingredients are added using techniques and at times in the manufacturing process as are known to those in the art. Desirably, any volatile components (such as fragrances) are added to the mixture at a relatively late stage of the mixing, so as to limit volatilization of the volatile components.

Generally, the method of making the antiperspirant and/or deodorant products of this invention may be described as follows. All of the non-volatile components including gellants (one or more of the polyamides and one or more of the co-gellants described above), the nonvolatile solvents (comprising silicone (for example, phenyltrimethicone or a suitable dimethicone), organics, and, optionally, organo-silicones and/or functionalized silicones as defined and described above) and any other non-volatile components (for example, additional organic emollients) are added to a vessel of suitable size. The mixture is heated to a temperature of about 120 degrees C. and melted and/or dissolved together with stirring. In a separate vessel, the antiperspirant active powder (which may be used in lower amounts if only a deodorant is desired) is mixed with the cyclomethicone or other volatile silicones as well as any other volatiles (for example, some of the isoparaffins) used and heated to a temperature of about 50 degrees C. with stirring. As noted above, the antiperspirant active is preferably added as a powder, but a premixed solution or slurry may also be used. If the antiperspirant active is premixed with a liquid first (for example dissolved in water and/or a water/glycol mixture), then the cyclomethicone and/or other volatiles are first added to the mixture of non-volatiles and then the antiperspirant solution or slurry is added to that mixture. The two mixtures (gellant mixture and actives mixture) (or three mixtures if a premix of antiperspirant active is used) are then combined with stirring for 10 minutes but without added heat. If colorant and fragrance are desired they may be added under agitation for another 5 minutes. The colorant may be added at any time, but the fragrance is preferably added as the last step to minimize loss. Mixing is continued with cooling. If it is desired to form packaged products, pouring usually takes place at a temperature in the range of 70-75 degrees C. into suitable containers (42.5 and 65.2 gram (1.4 oz or 2.3 oz) oval top containers). If these are stick compositions, they are allowed to solidify (for example, at room temperature or lower). It is preferred that any heating used in this process not be too long or too high because of adverse effects on the final product.

When a cosmetic composition according to the present invention is in the form of a stick product, the composition can be applied by elevating the stick out of the package so as to expose the end of the stick, and then rubbing the end of the stick on the skin in order to deposit stick material (including the cosmetically active material such as the antiperspirant active) on the skin. Thus, in the case of an antiperspirant, the active material on the skin is available to reduce body malodor and/or reduce the flow of perspiration from, for example, the axillary regions of the body.

In a series of preferred embodiments base compositions and cosmetic compositions according to the present invention contain a sufficient amount of the thickening agent such that the final cosmetic composition is a solid stick composition.

In the following, illustrative examples of compositions within the scope of the present invention are set forth. These examples are illustrative of the present invention, and are not limiting. Amounts of components in these examples are in weight percent, of the total weight of the composition.

Throughout the present specification, where compositions are described as including or comprising specific components or materials, or where methods are described as including or comprising specific steps, it is contemplated by the inventors that the compositions of the present invention also consist essentially of, or consist of, the recited components or materials, and also consist essentially of, or consist of, the recited steps. Accordingly, throughout the present disclosure any described composition of the present invention can consist essentially of, or consist of, the recited components or materials, and any described method of the present invention can consist essentially of, or consist of, the recited steps.

Throughout the specification and claims all percents are in percents by weight unless stated otherwise. If no standard is indicated, then the percent by weight is in reference the total weight of the cosmetic composition.

A translucent composition, although allowing light to pass through, causes the light to be scattered so that it will be impossible to see clearly objects behind the translucent composition. An opaque composition does not allow light to pass therethrough. Within the context of the present invention, a gel or stick is deemed to be transparent or clear if the maximum transmittance of light of any wavelength in the range 400-800 nm through a sample 1 cm thick is at least 35%, preferably at least 50%. The gel or stick is deemed translucent if the maximum transmittance of such light through the sample is between 2% and less than 35%. A gel or stick is deemed opaque if the maximum transmittance of light is less than 2%. The transmittance can be measured by placing a sample of the aforementioned thickness into a light beam of a spectrophotometer whose working range includes the visible spectrum, such as a Bausch & Lomb Spectronic 88 Spectro-photometer.

Residue may be evaluated by visual observation on the skin and fabric or by using more quantitative tests.

In the following, specific synthesis examples for forming compositions of this invention are set forth, specifically examples of antiperspirant and deodorant compositions. These specific synthesis examples and examples are illustrative in connection with the present invention, and are not limiting. In the following, as well as throughout the present disclosure, names utilized are the CTFA (Cosmetics, Toiletry and Fragrance Association, Inc.) names, as set forth in the CTFA International Cosmetic Ingredient Dictionary (4th Ed. 1991), the contents of which dictionary are incorporated herein by reference in their entirety. Throughout the description of this invention chemical abbreviations and symbols have their usual and customary meanings, temperatures are in degrees C., all percents are in weight percents based on the total weight of the composition, and comprising shall be interpreted as including as subgroups consisting of and consisting essentially of. While particular siloxane-based polyamides are disclosed or used in the following Examples, it is to be understood that other siloxane-based polyamides meeting the criteria of the invention may also be substituted for the particular siliconized polyamide used in the examples and such compositions are within the spirit and scope of the invention.

### EXAMPLES

### Example 1

### General Method

The following general manufacturing method is used to make a composition according to the present invention and using the following ingredients in the amounts indicated.

| Ingredient | Formula wt % | weight (g) |
|---|---|---|
| PPG-3 myristyl ether | 6 | 60 |
| Isostearyl alcohol | 10 | 100 |
| Siliconized polyamide | 9 | 90 |
| Dibutyl lauryl glutamide | 1 | 10 |
| Cyclomethicone | 49 | 490 |
| Westchlor 30BDM HBD | 25 | 250 |

For this method 60 g of PPG-3 myristyl ether and 100 g of isostearyl alcohol are weighed and placed into a 2 liter beaker. This mixture is than heated to 80° C. with constant agitation. Dibutyl lauryl glutamide (10 g) is added to this mixture and the entire mixture is heated to 120° C. until the dibutyl lauryl glutamide is dissolved. Ninety grams of a siliconized polyamide of Formula IIIA (with all the R groups being methyl, the average molecular weight being about 120,000 daltons and a DP=15) is then added to this mixture and dissolved. The temperature is held at 120° C. In a separate vessel, 490 g of D5 cyclomethicone is added into a 1 liter beaker. Two hundred and fifty grams of the antiperspirant active is then added to the cyclomethicone with constant agitation. This mixture is heated to 50° C. Thereafter the two mixtures are combined with stirring and no added heat. When the temperature of the entire system reaches around 75° C., it is poured into approximately 42.5 or 65.2 gram (1.4 oz or 2.3 oz) cosmetic product containers which are oval in cross-section.

### Examples 2-8

The method described in Example 1 is repeated with the types and amounts of ingredients listed below in TABLE A. For Examples 2-7, the antiperspirant active used was AZP Reheis 908-0. For Example 8, the antiperspirant active used was Westwood Westchlor 30 BDM HBD.

TABLE A

| Ingredient | Ex. 2 | Ex. 3 | Ex. 4 | Ex. 5 | Ex. 6 | Ex. 7 | Ex. 8 |
|---|---|---|---|---|---|---|---|
| Polyamide gellant | 9 | 9 | 9 | 10 | 10 | 9 | 9 |
| Dibutyl lauroyl | 0.7 | 1 | 1 | 1 | 1 | 0 | 1 |
| glutamide | | | | | | | |
| DBS | 0 | 0 | 0 | 0 | 0 | 0.25 | 0 |
| PPG-3 myristyl ether | 30 | 23 | 25 | 0 | 5 | 26.75 | 6 |
| Cyclomethicone (D5) | 35.3 | 30.5 | 35 | 35 | 54 | 34 | 49 |
| Colorant | 0 | 0.5 | 0 | 0 | 0 | 0 | 0 |
| Isostearyl alcohol | 0 | 11 | 5 | 10 | 0 | 5 | 10 |
| Dioctyl carbonate | 0 | 0 | 0 | 19 | 5 | 0 | 0 |
| Antiperspirant active | 25 | 25 | 25 | 25 | 25 | 25 | 25 |

### Examples 4 and 7

### Residue Evaluation

Compositions made according to Examples 4 and 7 were tested for white residue on skin and black fabric by rubbing samples on each surface. Comparisons were done with Lady Speed Stick Invisible Dry and SECRET Sheer Dry. FIGS. 1 and 2 are representative of the superior results obtained on black cloth with products of the invention vs. the two commercial products tested. For tests on skin, the results showed low white residue on skin. It should be noted that while commercial products have previously achieved low white residue on skin, it is the combination of low skin and low fabric residue that is an important result in this invention.

### Examples 9-14

The method of Example 1 may be repeated with polyamides of Formula IIIA with a molecular weight in the range of 90,000-120,000 and a DP in the range of 5-30 or 12-18 with R¹-R⁴ each being methyl. The antiperspirant active used is Reheis AZP 908-0.

TABLE B

| Ingredient | Ex. 9 | Ex. 10 | Ex. 11 | Ex. 12 | Ex. 13 | Ex. 14 |
|---|---|---|---|---|---|---|
| Polyamide gellant | 9 | 9 | 9 | 9 | 9 | 11 |
| Dibutyl lauroyl | 0.9 | 0 | 0 | 1.1 | 0 | .7 |
| glutamide | | | | | | |
| 12-hydroxy | 0 | 0.9 | 0 | 0 | 0 | 0 |
| stearic acid | | | | | | |
| Amine stearate | 0 | 0 | 1.2 | 0 | 0 | 0 |
| N,N′- | 0 | 0 | 0 | 0 | 1 | 0 |
| hexamethylene- | | | | | | |
| bis-(10-undecen- | | | | | | |
| amide) | | | | | | |
| PPG-3 myristyl | 20 | 23 | 25 | 28 | 23 | 26.75 |
| ether | | | | | | |
| Tween 60 | 1 | 1 | 1 | 1 | 1 | 1 |
| Cyclomethicone | 36.74 | 30.74 | 32.44 | 34.54 | 32.64 | 34.19 |
| (D5) | | | | | | |
| Fragrance | 1.2 | 1.2 | 1.2 | 1.2 | 1.2 | 1.2 |
| Isostearyl alcohol | 6 | 9 | 5 | 5 | 7 | 5 |
| Antiperspirant | 25 | 25 | 25 | 20 | 25 | 20 |
| active | | | | | | |
| BHT | .08 | .08 | .08 | .08 | .08 | .08 |
| Triethanolamine | .08 | .08 | .08 | .08 | .08 | .08 |

### Evaluation of Residue on Skin

The superior low white residue achievable with this invention can be evaluated in two ways, by panelists and by instruments. With panelists, a sample group such as a minimum of 24 people are used to evaluate products, in this case four products: a clear stick as a positive control, a regular white stick as a negative control, a prototype formula described in the patent as Example 12 (with DP=12-18), and a competitive formula (SECRET Sheer Dry). Panelists apply one product to each underarm at a time (4 swipes), and evaluate the appearance of the white residue on a 1 to 10 scale. The data is then analyzed using software such as JUMP for statistical analysis. A superior low-white-residue product should have an average evaluation score of 0.55 or less on skin as judged by the panelists. The Example of the type described in Example 12 with DP=12-18 was tested on a group of 24 Caucasian women and gave an average value of 0.33. SECRET Sheer Dry gave a value of 0 2.7. The regular white stick gave a value of 5.

In the method where instruments are used to measure the white residue of the formula, a Chroma meter, CR-300, made by Minolta, Japan can be used. The whiteness score of panelists' clean underarm is measured first on the instrment. Then the panelist applies a product to the underarm (4 swipes). The whiteness score of the underarm after the product application is then measured on the instrument. The difference of these two scores is the whiteness contributed by the product on the underarm. A superior low-white-residue product should have an average evaluation score of 0.55 or less. The average score from the measurements of the type described in Example 12 with DP=12-18 invention is 0.33 on the same group of 24 Caucasian women described above. The measurement on SECRET Sheer Dry was 0.74 and the measurment on the regular white stick gave a value of 2.10.

We claim:

1. A solid antiperspirant and/or deodorant composition exhibiting a low white residue on human skin of less than 0.55 as measured by a reflectometer on the composition applied to a human underarm which composition comprises:
(a) from 4-15% by weight based on the total weight of the composition of at least one siliconized polyamide of Formula IIIA as a primary gellant: 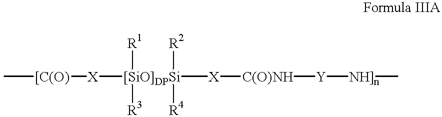
where:
(1) DP is a number in the range of 5-30;
(2) n is a number selected from the group consisting of 20-200;
(3) X is a linear or branched chain alkylene having 1-30 carbons;
(4) Y is selected from the group consisting of linear and branched chain alkylenes having 1-40 carbons, wherein:
(A) the alkylene group may optionally and additionally contain in the alkylene portion at least one of the members of a group consisting of (i) 1-3 amide linkages; (ii) C5 or C6 cycloalkane as a cycloalkylene linkage; and (iii) phenylene optionally substituted by 1-3 members selected independently from the group consisting of C1-C3 alkyls; and
(B) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y=Z² where 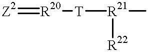
wherein each of R²⁰, R²¹ are independently selected from the group consisting of linear and branched C1-C10 alkylenes; R²² is selected from the group consisting of linear and branched C1-C10 alkanes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl; and
(5) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl;
wherein the polyamide of Formula IIIA has:
(i) a silicone portion in the acid side of the polyamide;
(ii) an average molecular weight of at least 10,000 daltons; and
(iii) a polydispersity of less than 20;
(b) a co-gellant which is up to 10 weight % of at least one member selected from the group consisting of N-acyl amino acid derivatives; dibenzylidene sorbitol; N,N′-hexamethylenebis-(10-undecenamide); amine stearate; 12-hydroxystearic acid; stearyl alcohol and waxes;
(c) a solvent system for the primary gellant and co-gellant in an amount of up to 90%; and
(d) a high density antiperspirant active having a bulk density of at least 0.45 g/cm³ and used in an amount to have a deodorant and/or antiperspirant effect;
wherein all amounts are in percent by weight based on the total weight of the composition.

2. A composition as claimed in claim 1 wherein the amount of co-gellant is in the range of 0.5-2.0% by weight based on the total weight of the composition.

3. A composition as claimed in claim 1 wherein the co-gellant comprises up to 5 weight % of one or more members selected from h group consisting of dibutyl lauroyl glutarnide; dibenzylidene sorbitol; N,N′-hexamethylenebis-(10-undecenamide); amine stearate; 12-hydroxystearic acid; stearyl alcohol and castor waxes.

4. A composition as claimed in claim 1 wherein the co-gellant is dibutyl lauroyl glutamide.

5. A composition as claimed in claim 1 wherein the average molecular weight of the polyamide is at least 30,000 daltons.

6. A composition as claimed in claim 1 wherein the average molecular weight of the polyamide is in the range of 80,000-150,000 daltons.

7. A composition as claimed in claim 6 wherein the average molecular weight of the polyamide is in the range of 80,000-90,000 daltons.

8. A composition as claimed in claim 6 wherein the average molecular weight of the polyamide is in the range of 90,000-120,000 daltons.

9. A composition as claimed in claim 1 wherein the polyamide has a polydispersity of less than 10.

10. A composition as claimed in claim 6 wherein the polyamide has a polydispersity of less than 10.

11. A composition as claimed in claim 8 wherein the polyamide has a polydispersity of less than 10.

12. A composition as claimed in claim 1 wherein the polyamide has a polydispersity of less than 4.

13. A composition as claimed in claim 6 wherein the polyamide has a polydispersity of less than 4.

14. A composition as claimed in claim 8 wherein the polyamide has a polydispersity of less than 4.

15. A composition as claimed in claim 1 wherein the DP is a number in the range of 12-18.

16. A composition as claimed in claim 6 wherein the DP is a number in the range of 12-18.

17. A composition as claimed in claim 8 wherein the DP is a number in the range of 12-18.

18. A composition as claimed in claim 1 wherein the DP is 15.

19. A composition as claimed in claim 6 wherein the DP is 15.

20. A composition as claimed in claim 8 wherein the DP is 15.

21. A composition as claimed in claim 1 wherein for the polyamide of Formula IIIA, R¹-R⁴ are each methyl.

22. A composition as claimed in claim 1 wherein the polyamide is a polyamide of Formula IIIB: 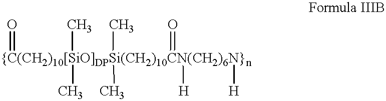
where DP is from 5-30 and n is a number in the range of 20-200 and is selected to give an average molecular weight of at least 10,000 daltons.

23. A composition as claimed in claim 22 wherein the polyamide has a molecular weight in the range of 90,000-120,000.

24. A composition as claimed in claim 22 wherein the DP is from 12-18.

25. A composition as claimed in claim 24 wherein the DP is 15.

26. A composition as claimed in claim 1 wherein for the polyamide of Formula IIIA, X, Y, DP and R¹-R⁴ remain the same in each polymeric unit.

27. A composition as claimed in claim 1 wherein the polyamide of Formula IIIA, contains multiple siloxane block lengths of Formula IIIC: 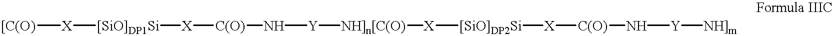
where X, Y, n, and R¹-R⁴ have the meanings described for Formula IIIA; m is selected from the same group as n, and n and m denote the total number of units enclosed within the brackets in a regular, alternating, block or random sequencing; R⁵-R⁸ is selected from the same group as defined for R¹-R⁴; DP1 and DP2 may be the same or different and are each independently selected from the same group as defined for DP; and the units denominated by n and m may be structured to form either block or random copolymers.

28. A composition as claimed in claim 27 wherein for block lengths of Formula IIIC, all of the R groups are methyl.

29. A composition as claimed in claim 27 wherein for block lengths of Formula IIIC, DP1=DP2.

30. A composition as claimed in claim 1 wherein the solvent system comprises one or more members selected from the group consisting of:
(1) from 5-65% by weight based on the total weight of the composition of at least one non-silicone organic selected from the group consisting of C12-36 esters; guerbet alcohols having 8-30 carbons; fatty alcohols having 8-30 carbons; ethoxylated and propoxylated alcohols having 3-30 carbons; alkyl ethers having 12-36 carbons; C12-18 alkyl benzoate and benzoate ester derivatives; paraffins having a distillation temperature in the range of 372-426 degrees C.; isoparaffins having a distillation temperature in the range of 178-207 degrees C.; and C6-30 alkyl carbonates.
(2) from 2-55% by weight based on the total weight of the composition of a volatile silicone selected from the group consisting of cyclomethicones and low viscosity dimethicones;
(3) from 0-10% organo-silicones; and
(4) from 0-40% of a functionalized silicone.

31. A composition as claimed in claim 30 wherein the solvent system comprises one or more members selected from the group consisting of: tridecyl neopentanoate, ethyl oleate, dioctyl carbonate, isopropyl myristate, octyl methoxycinnamate, PPG-14 butyl ether, PPG-3 myristyl ether, dioctyl ether, C12-15 alkyl benzoate, isostearyl benzoate, octyl dodecyl benzoate, octyl salicylate, dioctyl carbonate, octyldodecanol, and isostearyl alcohol.

32. A composition according to any one of claims 1-31 wherein the composition is an opaque suspension.

33. A composition according to any one of claims 1-31 which is made as an emulsion which is translucent to opaque.

34. A composition according to any one of claims 1-31 wherein the bulk density of the antiperspirant active is greater than 0.61 g/cm³.

35. A composition according to any one of claims 1-9 which is made as an emulsion and which additionally comprises as a solvent for the antiperspirant active a member of the group consisting of:
(a) ≦25 weight % water;
(b) ≦35 weight % of a glycol or polyglycol selected from the group consisting of ethylene glycol, propylene glycol, 1,2-propanediol, diethylene glycol, triethylene glycol, tetraethylene glycol, dipropylene glycol, tripropylene glycol, methyl propanediol, 1,6-hexanediol, 1,3-butanediol, 1,4-butanediol, PEG-4 through PEG-100, PPG-9 through PPG-34, pentylene glycol, neopentyl glycol, trimethylpropanediol, 1,4-cyclohexanedimethanol, 2,2-dimethyl-1,3-propanediol, 2,2,4,4-tetramethyl-1,3-cyclobutanediol; and
(c) ≦35 weight % of a water and glycol or polyglycol mixture where the glycol and polyglycol are selected from (b);
in an amount sufficient to dissolve the antiperspirant active.

36. A composition according to any one of claims 1-9 which is made as an emulsion and which additionally comprises as a solvent for the antiperspirant active a member of the group consisting of:
(a) ≦25 weight % water;
(b) ≦35 weight % of a glycol or polyglycol selected from the group consisting of propylene glycol, dipropylene glycol, tripropylene glycol, 2-methyl-1,3-propanediol, methyl propylene glycol, low molecular weight (less than 600) polyethylene glycol, low molecular weight (less than 600) polypropylene glycols; and
(c) ≦35 weight % of a water and glycol or polyglycol mixture where the glycol and polyglycol are selected from (b);
in an amount sufficient to dissolve the antiperspirant active.

37. A composition as claimed in claim 1 comprising at least one additional ingredient selected from the group consisting of volatile silicones, silicone gums, elastomers, polymethylmethacrylate, polyethylene, polypropylene, polytetrafluoroethylene emollients, colorants, antibacterial agents, inorganic particulates and fragrances.

38. A composition as claimed in claim 1 comprising 5-20% on an anhydrous basis of an antiperspirant active.
